(19) United States
(12) Patent Application Publication
(10) Pub. No.: US 2010/0305420 A1
(43) Pub. Date: Dec. 2, 2010

(54) FLEXIBLE CIRCUIT AND METHOD FOR FORMING THE SAME

(75) Inventor: Kenneth Curry, Oceanside, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/790,387

(22) Filed: May 28, 2010

Related U.S. Application Data
(63) Continuation-in-part of application No. 12/537,031, filed on Aug. 6, 2009.
Application No. 12/537,031 is a division of application No. 11/710,280, filed on Feb. 22, 2007, now Pat. No. 7,586,173.
(60) Provisional application No. 61/182,900, filed on Jun. 1, 2009.
(60) Provisional application No. 60/777,133, filed on Feb. 27, 2006.

(51) Int. Cl. A61B 5/1473; H01L 29/66; H01L 21/02

(52) U.S. Cl. 600/345; 257/414; 438/49; 257/E29.166; 257/E21.002

(57) ABSTRACT

A flexible circuit is provided herein that includes conductive material on the top and bottom planar surfaces of a dielectric substrate. The flexible circuit can be used in various applications, including use as a sensor. A via is used to provide electrical communication between the top and bottom surface of the flexible circuit. A method of preparing a flexible circuit and a medical instrument including the flexible circuit are also provided.

### RELATED APPLICATIONS

The present Application for Patent claims the benefit of provisional application Ser. No. 61/182,900, filed Jun. 1, 2009, and is a continuation-in-part application of U.S. patent application Ser. No. 12/537,031, filed Aug. 6, 2009, which is a divisional of U.S. application Ser. No. 11/710,280, filed Feb. 22, 2007, now U.S. Pat. No. 7,586,173, all which are assigned to the assignee hereof and the contents of which is hereby expressly incorporated by reference herein.

### FIELD OF THE DISCLOSURE

Flexible circuit technology is described herein and, more specifically, the creation and use of two-sided flexible circuits such as for sensors.

### BACKGROUND

Flexible circuits or “flex circuits” have been used in the micro-electronics industry for many years. Flex circuits are desirable due to their low manufacturing cost, ease in design integration, and use for various types of applications. In recent years, flex circuits have been used to design microelectrodes for sensors in in vivo applications. One flex circuit design involves a laminate of a conductive material and a flexible dielectric substrate. The flex circuit can be formed on the conductive foil using masking and photolithography techniques.

### SUMMARY

In a first embodiment, a method of creating a sensor is provided. The method comprises applying a first conductive material on a first portion of a substrate to form a reference electrode and depositing a first mask over the substrate, the first mask having an opening that exposes the reference electrode and a second portion of the substrate. The method can also include depositing a second conductive material into the opening in the first mask, the second conductive material being in direct contact with the reference electrode and depositing a second mask over the second conductive material, the second mask having an opening over the second portion of the substrate, the opening exposing a portion of the second conductive material, which forms a working surface to receive a fluid of interest.

In a second embodiment, a method of creating a sensor is provided. The method comprises applying a first conductive material on a first portion of a substrate to form a reference electrode and a second portion of the substrate to form a working electrode, and depositing a first mask on the substrate, the first mask having an opening that exposes the reference electrode, the working electrode, and an area between the reference electrode and the working electrode. The method may also include depositing a second conductive material on the reference electrode and in the area between the reference electrode and the working electrode and depositing a second mask on the second conductive material.

In a third embodiment, a “two-sided” flexible circuit such as for use in a sensor is provided that includes conductors on either side of a dielectric substrate that are electrically connected through the dielectric substrate. The flex circuit described herein can include wiring on either side of the dielectric substrate thereby allowing for a reduction of half or more of the width of the dielectric substrate and thus the flexible circuit. This allows the flex circuit when used as a sensor to be narrower when it is provided in a medical instrument such as a catheter or intraocular implant. Alternately, a flex circuit of standard width can be used that can include twice or more of the electrodes as have conventionally been used for the same flex circuit width.

In a fourth embodiment, a sensor including a flexible circuit is provided, comprising a flexible dielectric substrate having opposing first and second planar surfaces defining longitudinal, transverse and normal directions; one or more conductive contacts adjacent the first planar surface of the flexible dielectric substrate; one or more conductive contacts adjacent the second planar surface of the flexible dielectric substrate; a first dielectric mask adjacent the first planar surface and substantially covering the first planar surface, the first dielectric mask having one or more mask openings corresponding to one of more of the conductive contacts adjacent the first planar surface; a second dielectric mask adjacent the second planar surface substantially covering the second planar surface; at least one conductive material provided within the mask openings of the first dielectric mask and in electrical communication with the one or more conductive contacts adjacent the first planar surface; one or more membrane layers applied in physical contact with at least a portion of the conductive material; a via extending through the dielectric substrate and providing electrical communication between a contact adjacent the first planar surface and a contact adjacent the second planar surface to provide electrical communication between the conductive material within one of the mask openings and the contact adjacent the second planar surface; and wires in electrical communication with the contacts adjacent the first planar surface and the contacts adjacent the second planar surface. The one or more membrane layers can perform a chemical transduction that is communicated to the conductive material. For example, the one or more membrane layers can form a working electrode, a reference electrode and a counter electrode on the flex circuit and at least one of the working electrode, the reference electrode and the counter electrode can be in electrical communication with the via. The one or more membrane layers forming the working electrode can include a redox reactive species such as an enzyme for use in detecting glucose concentration.

In a first aspect of the fourth embodiment, the second dielectric mask includes one or more mask openings corresponding to one or more conductive contacts adjacent the second planar surface and further comprising a conductive material applied to the second dielectric mask adjacent the mask openings in the second dielectric mask such that the conductive material is in electrical connection with the one or more conductive contacts adjacent the second planar surface. In some embodiments, the conductive material can be applied to the second dielectric mask adjacent the mask openings in the second dielectric mask in electrical communication with two conductive contacts adjacent the second planar surface to form a thermistor with the two conductive contacts. In some embodiments, the sensor can further include a third dielectric mask adjacent the first dielectric mask and substantially covering the first dielectric mask, the third dielectric mask having one or more mask openings corresponding to one of more of the conductive contacts adjacent the first planar surface, at least a portion of the one or more membrane layers provided within the mask openings in the third dielectric mask. In some embodiments, the at least one contact and at least one membrane layer corresponding to the at least one contact are offset from one another such as in the transverse direction and are in communication with each other through the at least one conductive material provided in the mask openings of the first dielectric mask. In some embodiments, the at least one conductive material applied to at least one of the mask openings of the first dielectric mask is different than the conductive material applied to another of the at least one of the mask openings of the first dielectric mask.

The via provided with the flex circuit can be hollow or solid. In some embodiments, the via includes a layer of nickel and a layer of gold. In some embodiments, the via is formed by the conductive material applied to the mask openings in the first dielectric mask. The via can be directly below the conductive material with which it is in electrical communication.

In a fifth embodiment, a sensor is provided for measuring the concentration of a redox reactive species in a fluid of interest. The sensor includes a flexible dielectric substrate having opposing top and bottom planar surfaces defining longitudinal, transverse and normal directions; a working electrode comprising a membrane material including a redox reactive species and an underlying conductive material, the underlying conductive material in electrical communication with a conductive contact adjacent the top planar surface of the dielectric substrate; a counter electrode comprising a conductive material in electrical communication with a conductive contact adjacent the top planar surface of the dielectric substrate; a reference electrode comprising a conductive material in electrical communication with a conductive contact adjacent the top planar surface of the dielectric substrate; a bottom contact comprising a conductive material adjacent the second planar surface of the dielectric substrate; and a via extending in electrical communication with one of the working electrode, the counter electrode and the reference electrode and the bottom contact through the dielectric substrate along a normal direction to provide a conductive path between one of the working electrode, the counter electrode and the reference electrode and the bottom contact. The sensor can also include a first trace in electrical communication with the working electrode, a second trace in electrical communication with the counter electrode, and a third trace in electrical communication with the reference electrode, wherein the trace in electrical communication with the one of the working electrode, the counter electrode and the reference electrode that is in electrical communication with the bottom contact is provided adjacent the second planar surface of the dielectric substrate and the other traces are provided adjacent the first planar surface of the dielectric substrate.

In a first aspect of the fifth embodiment, a method for producing a flexible circuit is provided, comprising providing a substantially planar, flexible dielectric substrate having opposing first and second planar surfaces having longitudinal, transverse and normal directions; forming at least one first conductor layer adjacent the first planar surface of the dielectric substrate, the first conductor layer comprising one or more contacts and one or more wires; forming at least one second conductor layer adjacent the second planar surface of the dielectric substrate, the second conductor layer comprising one or more contacts and one or more wires; forming a hole in the normal direction through the first conductor, the dielectric substrate and the second conductor; depositing conductive material within the hole of the dielectric substrate to provide a conductive path extending through the dielectric substrate in a normal direction, wherein the conductive path is in electrical communication with the first conductor and the second conductor; forming a first dielectric mask adjacent the first planar surface and substantially covering the first planar surface, the first dielectric mask having one or more mask openings corresponding to the at least one first conductor; forming a second dielectric mask adjacent the second planar surface substantially covering the second planar surface; depositing at least one conductive material within the mask openings of the first dielectric mask in electrical communication with the at least one conductor adjacent the first planar surface; and depositing one or more membrane layers in physical contact with at least a portion of the conductive material. In some embodiments, depositing one or more membrane layers comprises depositing membrane layers to form a working electrode, a reference electrode and a counter electrode, wherein at least one of the working electrode, the reference electrode and the counter electrode is in electrical communication with the conductive path through the hole. Depositing one or more membrane layers can include depositing a membrane layer comprising a redox reactive species such as an enzyme for use in detecting glucose concentration and forming at least a portion of the working electrode.

In a second aspect, alone or in combination with anyone of the previous aspects of the fifth embodiment, forming a second dielectric mask includes forming a second dielectric mask comprising one or more mask openings corresponding to one or more contacts adjacent the second planar surface, the method further comprising applying a conductive material to the second dielectric mask adjacent the mask openings in the second dielectric mask such that the conductive material is in electrical connection with the one or more contacts adjacent the second planar surface. For example, the conductive material applied to the second dielectric mask adjacent the mask openings in the second dielectric mask can be in electrical communication with two conductive contacts adjacent the second planar surface and can form a thermistor with the two conductive contacts. In some embodiments, the method further includes forming a third dielectric mask adjacent the first dielectric mask and substantially covering the first dielectric mask, the third dielectric mask having one or more mask openings corresponding to one of more of the contacts adjacent the first planar surface, at least a portion of the one or more membrane layers provided within the mask openings in the third dielectric mask. In some embodiments, at least one contact and at least one membrane layer corresponding to the at least one contact are offset from one another such as in a transverse direction and are in communication with each other through the at least one conductive material provided in the mask openings of the first dielectric mask. In some embodiments, the at least one conductive material applied to at least one of the mask openings of the first dielectric mask is different than the conductive material applied to another of the at least one of the mask openings of the first dielectric mask.

In a third aspect, alone or in combination with anyone of the previous aspects of the fifth embodiment, the conductive material is deposited within the hole prior to forming the first dielectric mask and forming the second dielectric mask. The conductive material can be deposited within the hole of the dielectric substrate to form a hollow or a solid via. In some embodiments, the conductive material is deposited within the hole of the dielectric substrate by electroplating metal inside the hole. In some embodiments, the conductive material is deposited within the hole of the dielectric substrate by plating nickel via an electroless plating process and plating gold via an immersion plating process within the hole. In some embodiments, the at least one conductive material deposited within the mask openings of the first dielectric mask is deposited within the hole of the dielectric substrate to form a conductive path. In some embodiments, the at least one conductive material is deposited within the mask openings of the first dielectric mask by depositing at least one conductive material directly above the hole formed through the first conductor, the dielectric substrate and the second conductor.

In a sixth embodiment, a medical instrument such as a catheter is provided comprising a tubular body defining at least one lumen and a flexible circuit positioned in the tubular body. The flexible circuit can be as described herein in the aforementioned embodiments.

### BRIEF DESCRIPTION OF THE DRAWINGS

Reference will now be made to the accompanying drawings, which are not necessarily drawn to scale, and wherein:

FIG. 1 is a cross-section view of a reference electrode channel that is created using a flex circuit according to an embodiment disclosed herein;

FIG. 2 is a top view of a flex circuit according to an embodiment disclosed herein;

FIG. 3 is a top view of a mask that is used to cover the flex circuit shown in FIG. 2 according to an embodiment disclosed herein;

FIG. 4 is a top view showing a conductive material deposited into the opening of the mask according to an embodiment disclosed herein;

FIG. 5 is a top view of a mask that is used to cover a portion of the conductive material and the mask shown in FIG. 4 according to an embodiment disclosed herein;

FIG. 6 is a flow chart showing a method of creating the reference electrode channel of FIG. 1 according to an embodiment disclosed herein;

FIG. 7 is a cross-section view of a reference electrode channel that is created using a flex circuit according to an embodiment disclosed herein;

FIG. 8 is a top view of a flex circuit according to an embodiment disclosed herein;

FIG. 9 is a top view of a mask that is used to cover the flex circuit shown in FIG. 8 according to an embodiment disclosed herein;

FIG. 10 is a top view showing a conductive material deposited into the opening of the mask according to an embodiment disclosed herein;

FIG. 11 is a top view of a mask that is used to cover the conductive material and the mask shown in FIG. 10 according to an embodiment disclosed herein;

FIG. 12 is a flow chart showing a method of creating the reference electrode channel of FIG. 7 according to an embodiment disclosed herein;

FIG. 13 is an exploded view of the fabrication of a flex circuit according to one embodiment;

FIG. 14 is a side elevation view of a flex circuit along line 2-2 according to one embodiment after metal layers are applied to a dielectric substrate;

FIG. 15 is a side elevation view of a flex circuit along line 2-2 according to one embodiment after a hole is produced in the flex circuit;

FIG. 16 is a side elevation view of a flex circuit along line 2-2 according to one embodiment after a via is formed in the hole;

FIG. 17 is a side elevation view of a flex circuit along line 2-2 according to one embodiment after application of a bottom mask;

FIG. 18 is a side elevation view of a flex circuit along line 2-2 according to one embodiment after application of a thermistor;

FIG. 19 is a side elevation view of a flex circuit along line 2-2 according to one embodiment after application of a first top mask;

FIG. 20 is a side elevation view of a flex circuit along line 2-2 according to one embodiment after application of a conductive ink to the first top mask;

FIG. 21 is a side elevation view of a flex circuit along line 2-2 according to one embodiment after a second top mask is applied to the first top mask;

FIG. 22 is a side elevation view of a flex circuit along line 2-2 according to one embodiment after membrane layers are applied to the second top mask;

FIG. 23 is a side elevation view of a flex circuit along line 2-2 according to one embodiment after application of a polymeric material to the membrane layers;

FIG. 24 is a side elevation view of a flex circuit along line 2-2 according to one embodiment after application of a bottom mask;

FIG. 25 is a side elevation view of a flex circuit along line 2-2 according to one embodiment after application of a thermistor;

FIG. 26 is a side elevation view of a flex circuit along line 2-2 according to one embodiment after application of a first top mask;

FIG. 27 is a side elevation view of a flex circuit along line 2-2 according to one embodiment after application of a conductive ink to the first top mask;

FIG. 28 is a side elevation view of a flex circuit along line 2-2 according to one embodiment after a second top mask is applied to the first top mask;

FIG. 29 is a side elevation view of a flex circuit along line 2-2 according to one embodiment after membrane layers are applied to the second top mask;

FIG. 30 is a side elevation view of a flex circuit along line 2-2 according to one embodiment after application of a polymeric material to the membrane layers;

FIG. 31 is a plan view of the top surface of the flex circuit; and

FIG. 32 is a plan view of the bottom surface of the flex circuit.

### DETAILED DESCRIPTION

As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms. In the drawings and description, like numbers refer to like elements throughout.

In one embodiment, a flex circuit to create a reference electrode channel is provided. The flex circuit has a reference electrode that is masked and imaged onto a substrate. A first mask is deposited on the substrate. The first mask may have an opening that has a first end that exposes a portion of the reference electrode and a second end that exposes a portion of the substrate. The opening forms a reference electrode channel. A conductive material may be deposited into the opening of the first mask. A second mask is deposited on the first mask and the conductive material. The second mask may have an opening that exposes a portion of the conductive material that is over the substrate.

In another embodiment, a “two-sided” flexible circuit is provided herein that comprises conductive material on the top and bottom planar surfaces of a dielectric substrate. The flexible circuit can be used in various applications, including use as a sensor wherein electrodes are provided on one or more of the top and bottom surfaces of the flexible circuit. In some embodiments, the flexible circuit can be used as an amperometric sensor for continuous in vivo measurements of a variety of redox active chemical species. In particular, the flexible circuit can be used as an amperometric sensor for measuring redox active chemical species present in a fluid of interest such as a liquid biological sample (e.g. blood or urine).

The redox reactive species can include any compound capable of participating in a biological mechanism or otherwise reacting with another biological compound in a manner capable of causing electron transfer. The redox reactive species comprises a species reactive in a redox reaction (i.e., that is capable of being reduced and/or oxidized).

In some embodiments, the redox reactive species comprises a biomolecule. The term “biomolecule”, as used herein, refers to any chemical compound naturally occurring in a living organism. For example, the biomolecule can be an enzyme. Compounds possessing enzymatic activity can be used as many interactions including enzymes and their substrates result in a transfer of one or more electrons. One particular example is the glucose oxidase enzyme, which binds to glucose to aid in the breakdown thereof in the presence of water and oxygen into gluconate and hydrogen peroxide. Accordingly, in certain embodiments, the redox reactive species can include glucose oxidase or a glucose dehydrogenase, such as bacterial glucose dehydrogenase, which is a quinoprotein with a polycyclicquinone prosthetic group. Bacterial glucose oxidase can be obtained from various microorganisms such as Aspergillus species, e.g., Aspergillus niger (EC 1.1.3.4), type II or type VII. Bacterial glucose dehydrogenases can be obtained from various microorganisms, such as Acinetobacter calcoaceticus, Gluconobacter species (e.g., G. oxidans), and Pseudomonas species (e.g., P. fluorescens and P. aeruginosa). Alternatively, the redox reactive species can be a lactate oxidase or lactate hydrogenase.

Many oxidases exhibit redox reactivity arising from the presence of a co-factor, such as flavin adenine dinucleotide (FAD). Thus, in certain embodiments, the redox reactive species comprises an FAD-containing oxidase enzyme. The flavin group of FAD is capable of undergoing redox reactions accepting either one electron in each step of a two-step process or accepting two electrons at once. In the reduced forms (e.g., FADH and FADH₂), the flavin adenine dinucleotide compound is capable of transferring electrons to other compounds or conductive materials. Non-limiting examples of FAD-containing enzymes that can be used include glucose oxidase, lactate oxidase, monoamine oxidase, D-amino acid oxidase, xanthine oxidase, and Acyl-CoA dehydrogenase. In some embodiments, the sensor is a glucose biosensor and the membrane includes a FAD-containing oxidase enzyme as the redox reactive species.

In some embodiments, the enzyme is an oxidase enzyme and/or a flavin adenine dinucleotide (FAD) containing enzyme. For example, the enzyme can include a FAD-containing glucose oxidase enzyme. The enzyme can be provided in particulate form such as a lyophilized powder.

The flexible circuit can allow for detection and measurement of virtually any redox active chemical species present within a sample. This specifically extends to in vivo measurements of various compounds present in living subjects. Accordingly, the redox reactive species present in the membrane can be any compound capable of coupling with another compound (such as another species) in a redox reaction. For clarity, the example of glucose oxidase reacting with glucose is described herein although other analytes can be measured. Thus, the membrane can be customized for use in electrochemically detecting and measuring any analytes produced or otherwise present within a living subject by selecting the appropriate redox reactive species that will interact with the analyte of interest in a redox reaction. This includes not only enzyme/substrate interactions but also encompasses other biochemical interactions.

FIG. 1 is a cross-section view of a reference electrode channel that is created using a flex circuit according to an embodiment disclosed herein. The flex circuit 102 may include a substrate 108, a trace 120, and a reference electrode 125. The trace 120 and the reference electrode 125 may be masked and imaged onto the substrate 105. For example, the trace 120 and the reference electrode 125 may be formed on the substrate 105 using screen printing or ink deposition techniques. The trace 120 and the reference electrode 125 may be made of a carbon, copper, gold, graphite, platinum, silver-silver chloride, rhodium, or palladium material.

A first mask 130 may be applied or deposited over a portion of the substrate 108 and over the trace 120. The first mask 130 may have an opening 135 that expose a portion of the reference electrode 125 and a portion of the substrate 108. The opening 135 forms the reference electrode channel. A conductive material 140 is deposited in the opening 135 to cover the exposed portion of the reference electrode 125 and the exposed portion of the substrate 108. A second mask 150 may be applied or deposited over the first mask 130 and the conductive material 140. The second mask 150 may have an opening 160 over a portion of the conductive material 140 that is over the substrate 108. The opening 135 is positioned along a first axis or plane and the opening 160 is positioned along a second axis or plane. The first axis or plane is not coincident with the second axis or plane. Hence, the first axis or plane is vertically and/or horizontally offset from the second axis or plane.

The opening 160 is the measurement site and allows a fluid of interest (e.g., blood, urine, etc.) to come into contact with the conductive material 140 to complete the measurement circuit with another measuring electrode (not shown) in contact with the same fluid. The conductive material 140 stabilizes the reference potential in several ways. The conductive material 140 may provide known silver and chloride ion activity, for example, (in the case of a silver-silver chloride reference design) to maintain a stable potential. The conductive material 140 should offer sufficient diffusion resistance to inhibit loss of desired ions to the fluid of interest, while simultaneously inhibiting migration of unwanted ions toward the active surface of the reference electrode 125. Spacing the opening 160 a sufficient distance from the reference electrode 125, as shown in FIG. 1, enhances this diffusion resistance. Finally, the conductive material 140 may provide a predictable junction potential at the interface with the fluid of interest which facilitates accurate electrochemical measurements using the reference electrode 125.

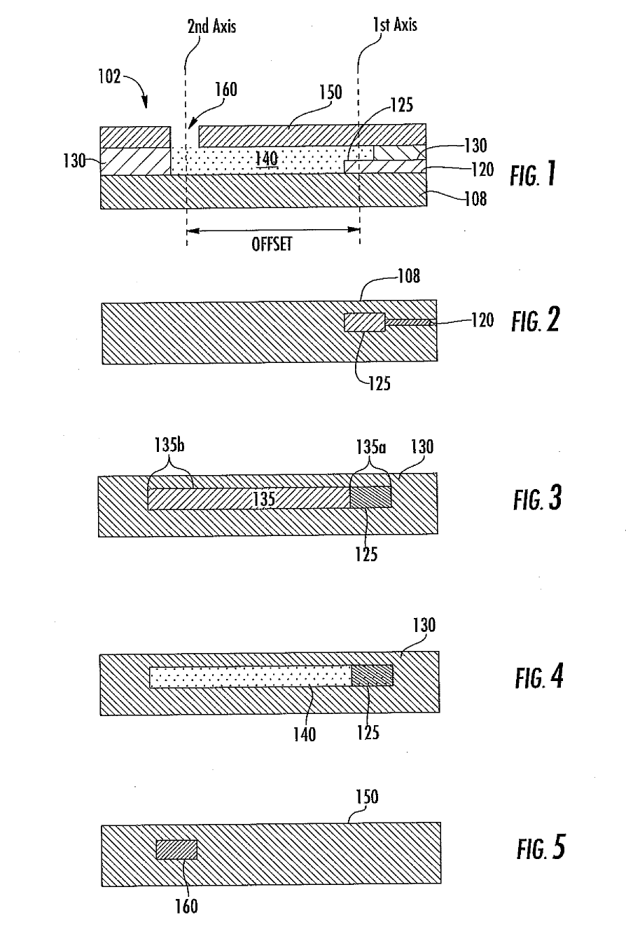

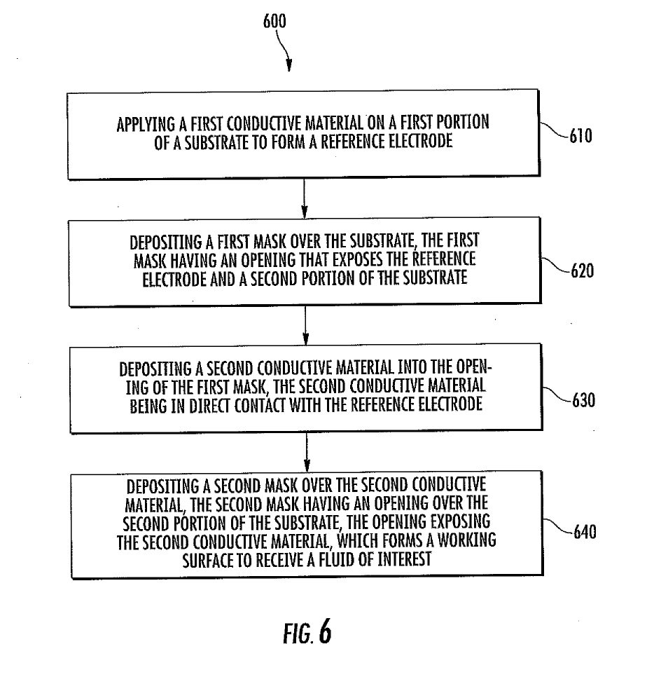

FIG. 2 is a top view of a flex circuit 102 according to an embodiment disclosed herein. The trace 120 and the reference electrode 125 may be made of a conductive material such as a silver-silver chloride (Ag/AgCl) material and may be formed on the substrate 108 using photolithography or printing techniques (610). For example, the trace 120 and the reference electrode 125 may be formed on the substrate 108 using screen printing or ink deposition techniques. The substrate 108 may be a flexible dielectric substrate such as a polyimide. The trace 120 may be used to connect to a measurement device (not shown) such as a potentiostat. The trace 120 is used to measure a potential from the reference electrode 125 using the measurement device. Even though FIG. 1 shows the flex circuit 102 having one trace 120 and one reference electrode 125, the flex circuit 102 may have more than one trace and more than one electrode.

FIG. 3 is a top view of a mask 130 that is used to cover the flex circuit 102 shown in FIG. 2 according to an embodiment disclosed herein. The mask 130 may be made of a dielectric material such as a photoimagable epoxy or an ultraviolet curable epoxy material. The mask 130 is deposited over the substrate 108 and has a rectangular opening 135 that has a first end 135a that exposes a portion of the reference electrode 125 and a second end 135b that exposes a portion of the substrate 108 (620). The rectangular opening 135 may have a length of between about 0.10-0.20 inches and a width of between about 0.010-0.020 inches. The length-to-width ratio of the rectangular opening 135 may be in the range of between about 4:1 to 12:1. In one embodiment, the mask 130 covers the entire top surface of the flex circuit 102 except for the rectangular opening 135. The mask 130 may have a thickness of between about 0.005 inches and about 0.02 inches. The first end 135a of the opening 135 is positioned directly above the electrode 125 so that the electrode 125 is exposed or visible through the opening 135 of the mask 130. Lithography techniques may be used to deposit or place the mask 130 on the flex circuit 102.

FIG. 4 is a top view showing a conductive material 140 deposited into the opening 135 of the mask 130 according to an embodiment disclosed herein. The conductive material 140 is deposited in the opening 135 to cover and to come into direct contact with the exposed portion of the reference electrode 125 and the exposed portion of the substrate 108 (630). The conductive material 140 may be a conductive fluid, a conductive solution, a conductive gel, a salt containing gel, a conductive polymer containing potassium chloride (KCl) with a small amount of silver ion (Ag⁺), or a material having conductive properties. For the case of a silver-silver chloride reference electrode 125, addition of a trace of silver nitrate solution to a matrix containing potassium chloride precipitates some amount of silver chloride within the conductive matrix, but maintains a silver ion concentration at a constant amount according to the solubility product of silver chloride, which is 1.56×10⁻¹⁰.

FIG. 5 is a top view of a mask 150 that is used to cover a portion of the conductive material 140 and the mask 130 shown in FIG. 4 according to an embodiment disclosed herein. The mask 150 may be made of a dielectric material such as a photoimagable epoxy or an ultraviolet curable epoxy material. The mask 150 has an opening 160 that exposes a portion of the conductive material 140 that forms a working surface to receive a fluid of interest (640). Lithography techniques may be used to deposit or place the mask 150 on the mask 130 and the conductive material 140. FIG. 6 shows a flow chart of the method of creating the reference electrode channel corresponding to FIGS. 1-5 as described above.

FIG. 7 is a cross-section view of a reference electrode channel that is created using a flex circuit according to an embodiment disclosed herein. The flex circuit 200 may include a substrate 210, traces 220 and 230, a reference electrode 225, and a working electrode 235. The traces 220 and 230, the reference electrode 225, and the working electrode 235 may be masked and imaged onto the substrate 210. For example, the traces 220 and 230, the reference electrode 225, and the working electrode 235 may be formed on the substrate 210 using screen printing or ink deposition techniques. The traces 220 and 230, the reference electrode 225, and the working electrode 235 may be made of a carbon, copper, gold, graphite, platinum, silver-silver chloride, rhodium, or palladium material.

A first mask 240 may be applied or deposited over a portion of the substrate 210 and over the traces 220 and 230. The first mask 240 may have an opening 250 that expose a portion of the reference electrode 225, a portion of the working electrode 235, and a portion of the substrate 210. The term “channel” (shown as channel 255) may be used to refer to the portion between the reference electrode 225 and the working electrode 235. Hence, the opening 250 may form the reference electrode channel. A conductive material 260 is deposited in the opening 250 to cover and to come into direct contact with the exposed portion of the reference electrode 225 and up to the edge of the exposed portion of the substrate 210. A second mask 265 may be applied or deposited over the first mask 240 and the conductive material 260. The second mask 265 may have an opening 270 over a portion of the working electrode 235. The reference electrode 225 is positioned along a first axis or plane and the working electrode 235 is positioned along a second axis or plane. The first axis or plane is not coincident with the second axis or plane. Hence, the first axis or plane is vertically and/or horizontally offset from the second axis or plane.

The opening 270 is the measurement site and allows a fluid of interest (e.g., blood, urine, etc.) to come into contact with the working electrode 235 and the conductive material 260 for a more accurate measurement. The conductive material 260 stabilizes the reference potential in several ways. The conductive material 260 may provide known silver and chloride ion activity for example (in the case of a silver-silver chloride reference design) to maintain a stable potential. The conductive material 260 should offer sufficient diffusion resistance to inhibit loss of desired ions to the solution, while simultaneously inhibiting migration of unwanted ions toward the active surface of the reference electrode 225. Spacing the opening 270 a sufficient distance from the reference electrode 225, as shown in FIG. 7, enhances this diffusion resistance. In addition, the opening 270 communicates directly with the end of the conductive material 260 at a smaller opening 275. The proximity of the smaller opening 275 to the working electrode 235 makes this embodiment ideal for situations where the solution resistance between the reference electrode and the working electrode needs to be keep at a minimum, such as in the case of a 3-electrode amperometric cell, for example.

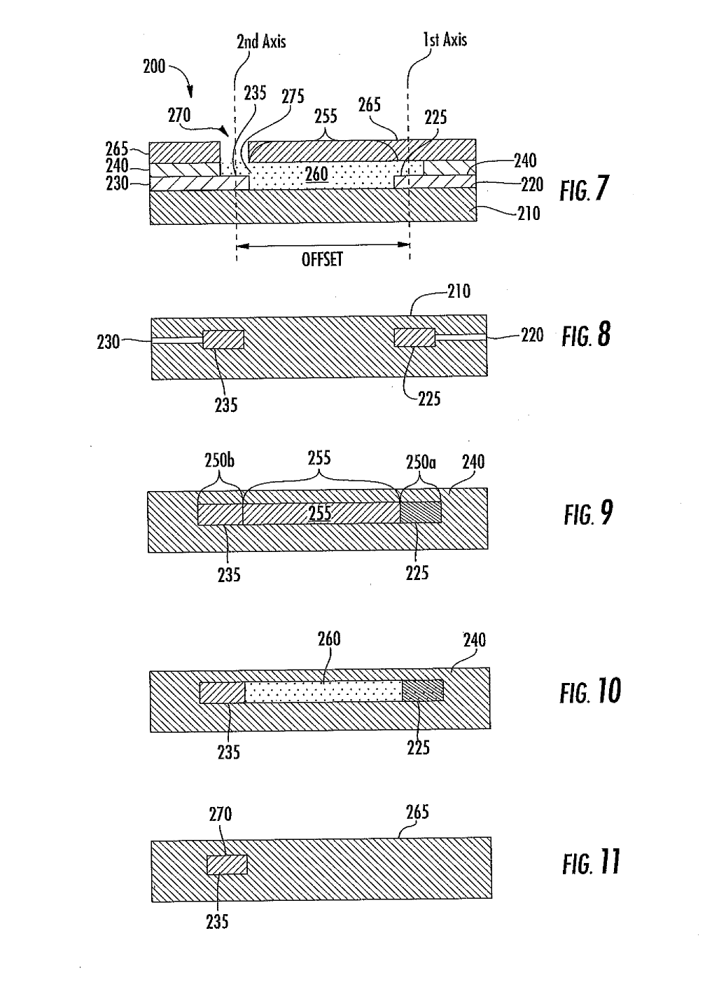

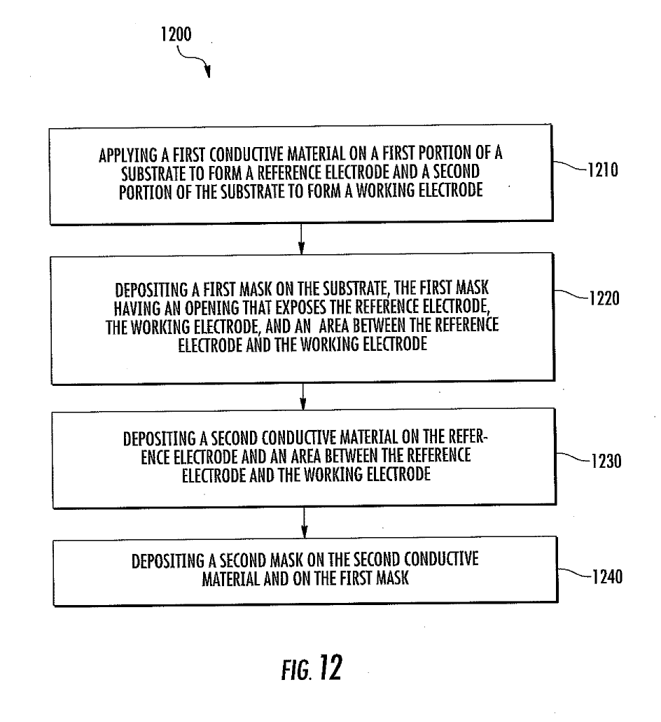

FIG. 8 is a top view of a flex circuit 200 according to an embodiment disclosed herein. The traces 220 and 230, the reference electrode 225 and the working electrode 235 may be made of a conductive material such as a copper material, a platinum material, a silver-silver chloride (Ag/AgCl) material and are formed on the substrate 210 using masking and photolithography techniques (1210). For example, the traces 220 and 230, the reference electrode 225, and the working electrode 235 may be formed on the substrate 210 using screen printing or ink deposition techniques. The substrate 210 may be a flexible dielectric substrate such as a polyimide. The traces 220 and 230 may be used to connect to a measurement device (not shown) such as a potentiostat. The traces 220 and 230 may be used to carry voltage or current from the reference electrode 225 and the working electrode 235 to the measurement device.

FIG. 9 is a top view of a mask 240 that is used to cover the flex circuit 200 shown in FIG. 8 according to an embodiment disclosed herein. The mask 240 may be made of a dielectric material such as a photoimagable epoxy or an ultraviolet curable epoxy material. The mask 240 is deposited over the substrate 210 and has a rectangular opening 250 that has a first end 250a that exposes a portion of the reference electrode 225, a second end 250b that exposes a portion of the working electrode 235, and a channel or an area 255 between the reference electrode 225 and the working electrode 235 that exposes a portion of the substrate 210 (1220). The rectangular opening 250 may have a length of between about 0.10-0.20 inches and a width of between about 0.010-0.020 inches. The length-to-width ratio of the rectangular opening 250 may be in the range of between about 4:1 to 12:1. In one embodiment, the mask 240 covers the entire top surface of the flex circuit 210 except for the rectangular opening 250. The mask 240 may have a thickness of between about 0.005 inches and about 0.02 inches. In one embodiment, the first end 250a of the opening 250 is positioned directly above the reference electrode 225 so that the reference electrode 225 is exposed or visible through the opening 250 of the mask 240. In one embodiment, the second end 250b of the opening 250 is positioned directly above the working electrode 235 so that the working electrode 235 is exposed or visible through the opening 250 of the mask 240. Lithography techniques may be used to deposit or place the mask 240 on the flex circuit 200.

FIG. 10 is a top view showing a conductive material 260 deposited into the opening 250 of the mask 240 according to an embodiment disclosed herein. The conductive material 260 is deposited in the opening 250 to cover and to come into direct contact with the exposed portion of the reference electrode 225 and in the area 255 between the reference electrode 225 and the working electrode 235 (i.e., on the exposed portion of the substrate 210) (1230). In one embodiment, a screenable gel or a conductive polymer is applied in the opening 250 to cover and to come into direct contact with the exposed portion of the reference electrode 225 and in the area 255 between the reference electrode 225 and the working electrode 235. The conductive material 260 may be a conductive fluid, a conductive solution, a conductive gel, a salt containing gel, a conductive polymer containing potassium chloride (KCl) with a small amount of silver ion (Ag⁺), or a material having conductive properties. The conductive material 260 may form a salt channel or a reference electrode channel.

FIG. 11 is a top view of a mask 265 that is used to cover the conductive material 260 and the mask 240 shown in FIG. 10 according to an embodiment disclosed herein. The mask 265 may be made of a dielectric material such as a photoimagable epoxy or an ultraviolet curable epoxy material. The mask 265 has an opening 270 that exposes a portion of the working electrode 235 and an edge of the conductive material 260, which forms a space to receive a fluid of interest. Lithography techniques may be used to deposit or place the mask 265 on the mask 240 and the conductive material 260 (1240). FIG. 12 shows a flow chart of the method of creating the reference electrode channel corresponding to FIGS. 7-11 as described above.

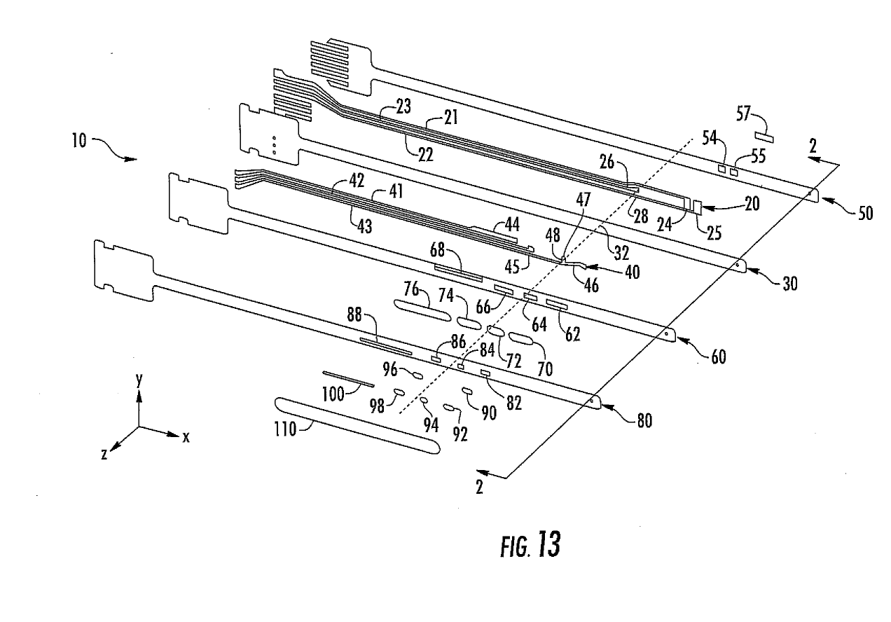

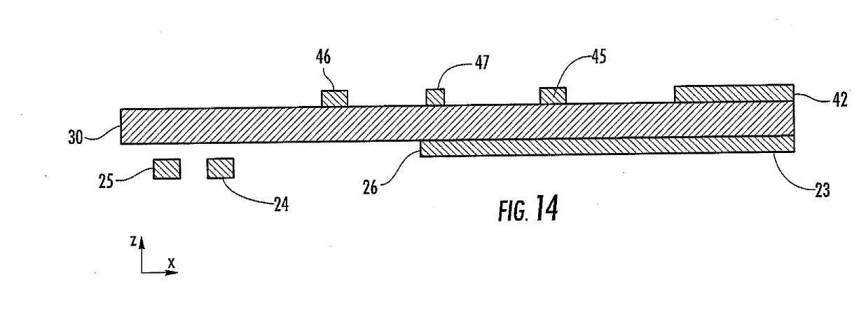

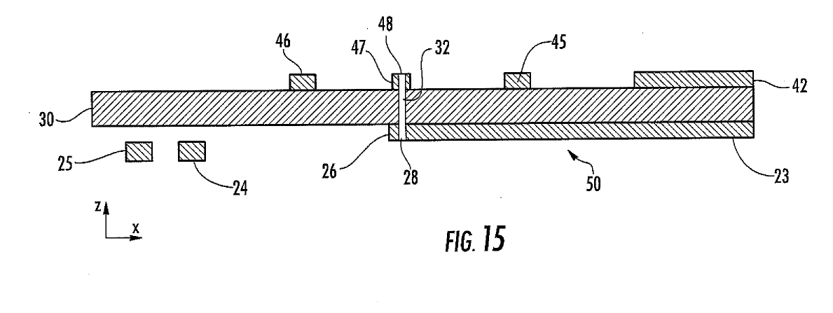

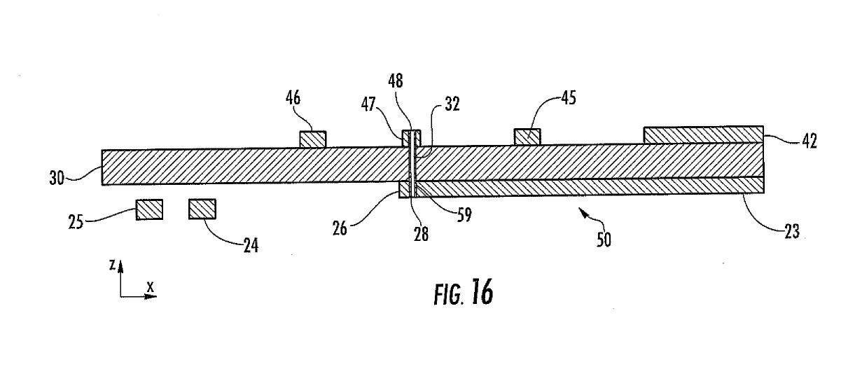

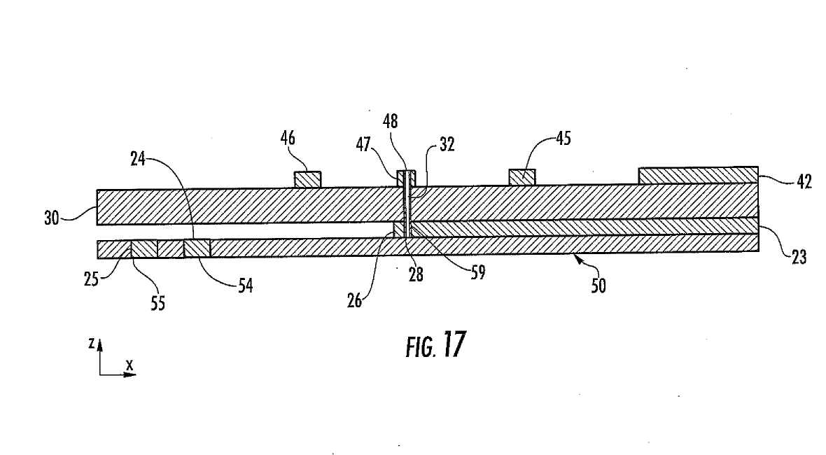

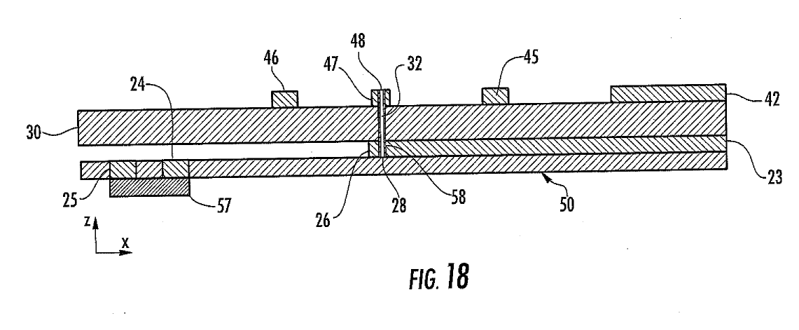

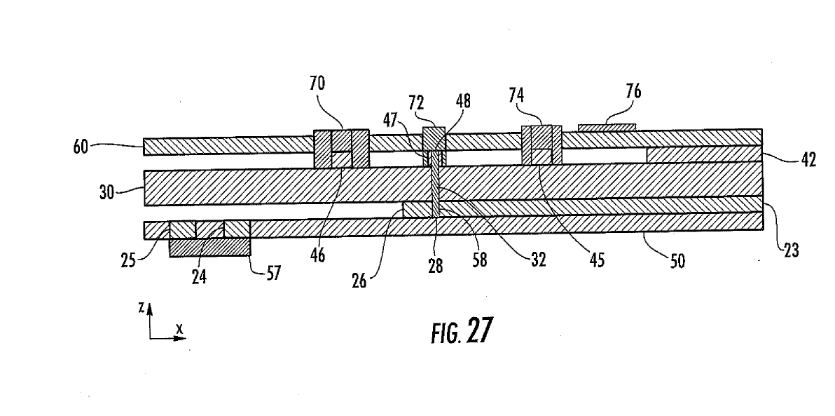

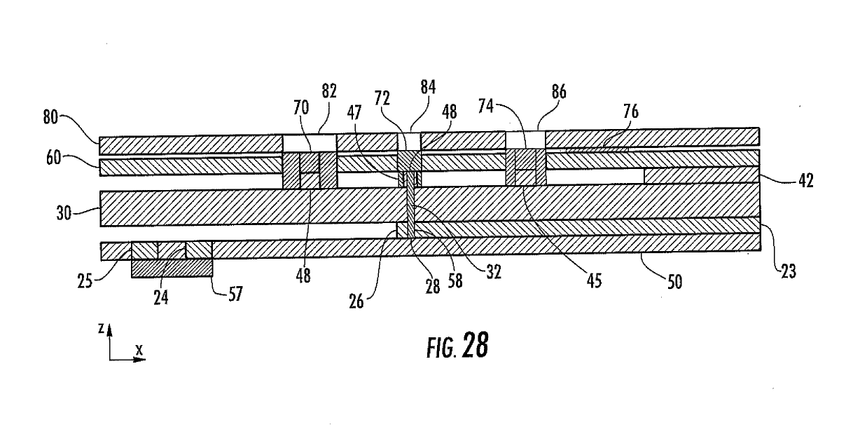

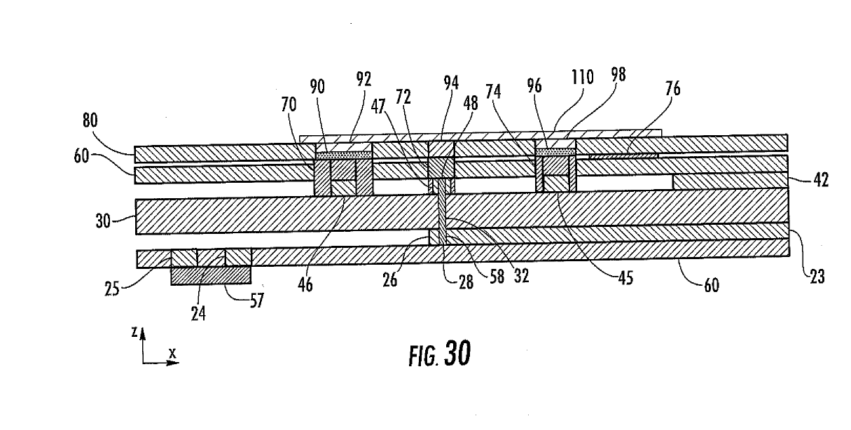

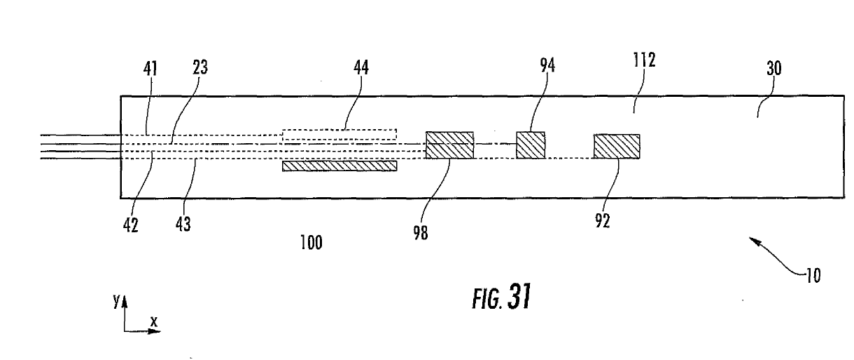

In one embodiment, a “two-sided” flexible circuit is provided herein that comprises conductive material on the top and bottom planar surfaces of a dielectric substrate. The flex circuit can be formed using masking and lithography techniques known in the art. FIGS. 13-30 illustrate exemplary methods for forming the flex circuit. FIG. 13 is an exploded view of the fabrication of a two-sided flexible circuit 10 or “flex circuit” according to an exemplary embodiment. The length of the flex circuit 10 defines an x-axis or horizontal axis in a longitudinal direction and the width of the flex circuit defines a y-axis or vertical axis in a transverse direction. The layers of the flex circuit 10 are applied along a z-axis in a normal direction. In some embodiments, the flex circuit 10 can have a generally rectangular shape.

As shown beginning in FIG. 13, the flex circuit is fabricated first by providing a bottom metal layer 20 and a top metal layer 40 on a dielectric substrate 30. The metal layers 20 and 40 can be formed of conductive materials such as carbon, gold, graphite, platinum, silver-silver chloride, rhodium, palladium, other metals, or other materials having specific electrochemical properties. In some embodiments, the bottom metal layer 20 and the top metal layer 40 can independently be formed of a highly conductive metal such as copper, platinum, or a combination thereof. In some embodiments, both the bottom metal layer 20 and the top metal layer 40 are formed of copper or a copper alloy. The top and bottom metal layers 20 and 40 can be formed using standard microfabrication processes known in the art such as screen or ink jet printing, microlithography, photolithography, electroplating, vapor deposition, or other metal deposition methods.

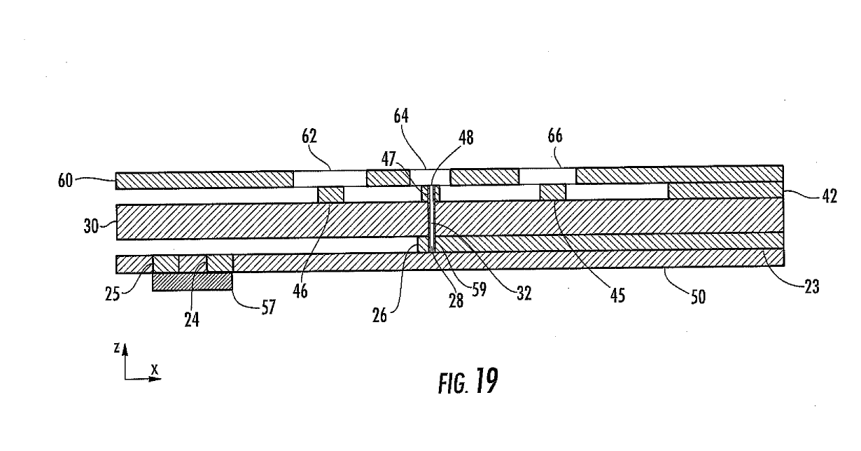

As shown in FIG. 13, the bottom metal layer 20 can include wires or traces 21, 22 and 23 that are provided along at least a portion and generally a substantial portion of the length of the dielectric substrate 30. The wires 21, 22 and 23 can communicate with contacts 24, 25 and 26, respectively. Although the contacts 24, 25 and 26 are illustrated as tabs having a width greater than the width of a wire, a contact can also be a portion of a wire. FIG. 14 illustrates wire 23 in electrical communication with contact 26, which are both present along centerline 2-2. By being in electrical communication, it is meant that there is a conductive path for electrons between the wire 23 and the contact 26 that exists even when the wires of the flex circuit are not connected to a measurement device such as a potentiostat. The contacts 24 and 25 can be connected to a measurement device through wires 21 and 22, which can carry voltage or current from the measurement device to the contacts to form a circuit. As shown in FIG. 14, the contacts 24 and 25 can be displaced from the dielectric substrate 30 in the z-direction such that they do not directly contact but are adjacent to the dielectric substrate 30.

The dielectric substrate 30 can be formed of any suitable insulative material. In some embodiments, the dielectric substrate 30 is a polymeric material such as a polyimide material. In some embodiments, the dielectric can be a flexible material.

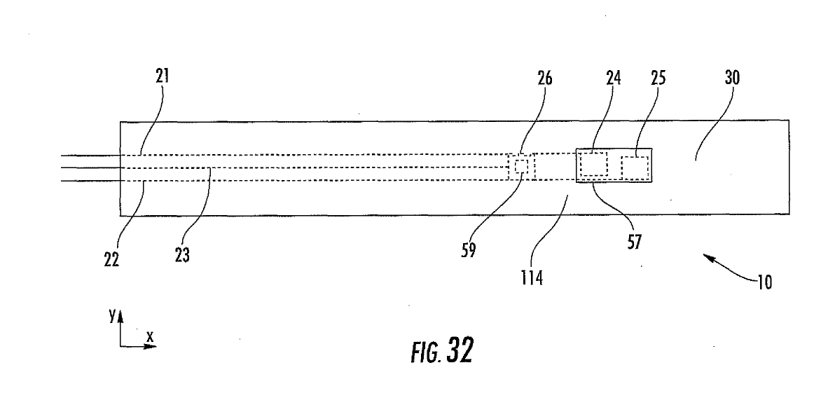

The top metal layer 40 can include wires or traces 41, 42 and 43 that are provided along at least a portion and generally a substantial portion of the length of the dielectric substrate 30. The wires 41, 42 and 43 can be in electrical communication with contacts 44, 45 and 46, respectively. As with the bottom metal layer 20, the contacts 44, 45 and 46 of the top metal layer 40 can be connected to a measurement device such as a potentiostat through wires 41, 42 and 43, which can carry voltage or current from the measurement device to the contacts. The top metal layer 40 can also include a contact 47. It is noted that FIG. 14 does not illustrate metal contact 44 as it is offset in the y-direction (or transverse direction) from the centerline defined by line 2-2. Metal contact 44 is illustrated, however, in FIGS. 13 and 31.

As shown in FIG. 15, a small hole can be formed in the flex circuit 10 as shown by the holes 28, 32 and 48 formed within the bottom metal layer 20, the dielectric substrate 30, and the top metal layer 40, respectively. As a result, the holes 28, 32 and 48 are aligned in the z-direction. The holes 28, 32 and 48 can be formed, for example, by physical or laser drilling, punching, or stamping. Alternatively, the hole 32 can be formed in the dielectric substrate 30 using these methods prior to depositing the metal layers 20 and 40 and the holes 28 and 48 can be formed in the metal layers 20 and 40 by suitable means such as etching.

In some embodiments, as shown in FIG. 16, a conductive material can be deposited in holes 28, 32 and 48 to form a via 59. The conductive material can be, for example, a bi-layer of nickel and gold. Conventional lithography techniques can be utilized to assure plating only within the holes 28, 32 and 48. For example, the conducting material can be applied via plating, such as electroless plating of the nickel and subsequent immersion plating of the gold. FIG. 16 illustrates a hollow via 59 wherein conductive material is deposited around the perimeter of the holes 28, 32 and 48 to form the via 59. Nevertheless, the via 59 can be solid by completely filling the holes 28, 32 and 48 with a conductive material. For example, a conductive material such as graphite can be blown into the holes 28, 32 and 48, a vacuum applied, and the graphite baked to form a solid via.

As shown in FIG. 17, a bottom mask 50 can be provided adjacent the bottom metal layer 20 using, e.g., conventional lithography techniques. For example, the bottom mask 50 can be applied in blanket form and then lithographically patterned by removing the material to form openings, such as openings 54 and 55. The bottom mask 50 can be made of a dielectric material, such as a photoimagable epoxy material or an ultraviolet (UV) curable epoxy material and can have a thickness of between about 0.005 inches and about 0.02 inches. The openings 54 and 55 can correspond to contacts 24 and 25 as shown in FIG. 17.

As shown in FIG. 18, a thermistor 57 can be provided onto the bottom mask 50 such that it is in electrical communication with contacts 24 and 25 that are provided in openings 54 and 55 of the bottom mask 50. The thermistor 57 can be formed of a conductive material such as a conductive epoxy material (e.g. a silver filled epoxy material). In some embodiments, the thermistor 57 is adhered to the bottom mask 50. The thermistor 57 works as a resistor having resistance that varies with temperature thus allowing the sensor to detect changes and temperature and any measurements made by the sensor can be modified accordingly.

As shown in FIG. 19, a first top mask 60 can be applied adjacent the top metal layer 40 using conventional lithography techniques such as applying the first top mask 60 in blanket form and lithographically patterning the first top mask 60 to form openings such as openings 62, 64, 66 and 68. The first top mask 60 can be made of a dielectric material, such as a photoimagable epoxy material or an ultraviolet (UV) curable epoxy material. The openings 62, 64, 66 and 68 can correspond to underlying metal contacts 46, 47, 45 and 44, respectively. In particular, opening 62 surrounds and corresponds with contact 46, opening 66 surrounds and corresponding to contact 45, and opening 68 corresponds to contact 44. Opening 68 can have substantially the same profile as contact 44. As opening 68 is offset in the y-direction from the centerline 2-2 like contact 44, it is not illustrated in FIG. 19. As shown in FIG. 19, opening 64 can surround and be aligned with the underlying metal contact 47 and via 59.

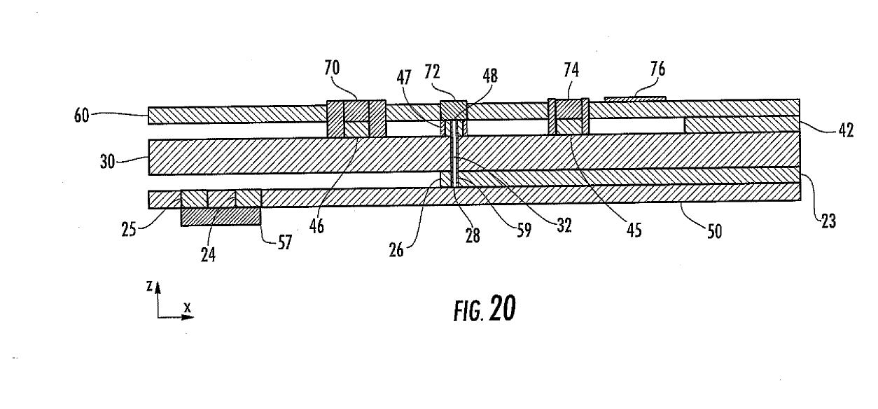

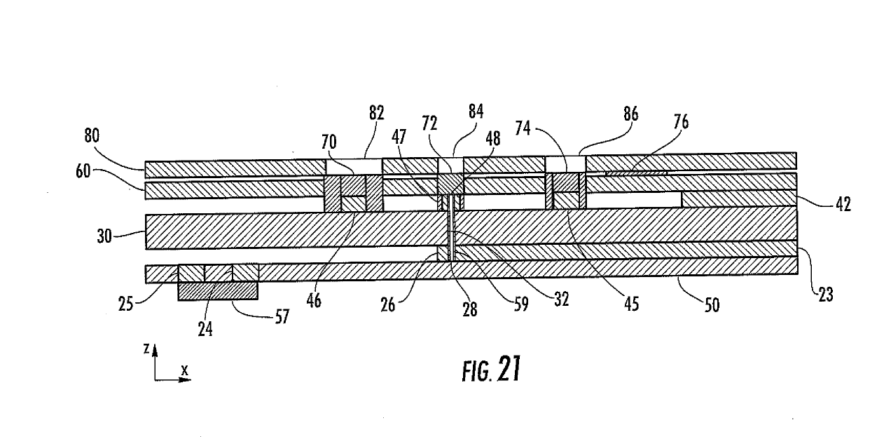

Conductive inks can be applied to openings 62, 64, 66 and 68 in the first top mask 60 as illustrated in FIG. 20. Specifically, conductive ink layers 70, 74 and 76 can be applied within openings 62, 66 and 68 respectively and cover underlying metal contacts 46, 45 and 44, respectively. A conductive ink layer 72 can also be applied through opening 64 to correspond to via 59. Although the conductive ink layer 72 is illustrated as being generally above via 59, a portion of the conductive ink could fill at least a portion of the via if the via is hollow depending on the size of the hole within the via and the viscosity and cohesive and adhesive properties of the conductive ink. The conductive ink layers 70, 72, 74 and 76 can include the same or different conductive material and can be applied by screen or ink jet printing, microlithography, photolithography, electroplating, vapor deposition or other methods. The conductive ink can be a conductive fluid, a conductive solution, a conductive gel, or a salt containing gel, and, in some embodiments, can be a platinum/graphite ink or a silver/silver chloride ink. In some embodiments, the conductive ink is applied by screen printing. The conductive ink layers 70, 72, 74 and 76 provide at least a portion of the conductive material that transmits electrons from the electrodes as described in more detail herein.

As illustrated in FIG. 21, a second top mask 80 having openings 82, 84, 86 and 88 can be applied to the top surface of the flex circuit 10 using conventional lithography techniques such as applying the second top mask 80 in blanket form and lithographically patterning the second top mask 80 to produce the openings. The second top mask 80 can be made of a dielectric material, such as a photoimagable epoxy material or an ultraviolet (UV) curable epoxy material. Openings 82, 84, 86 and 88 can correspond to conductive ink layers 70, 72, 74 and 76, respectively.

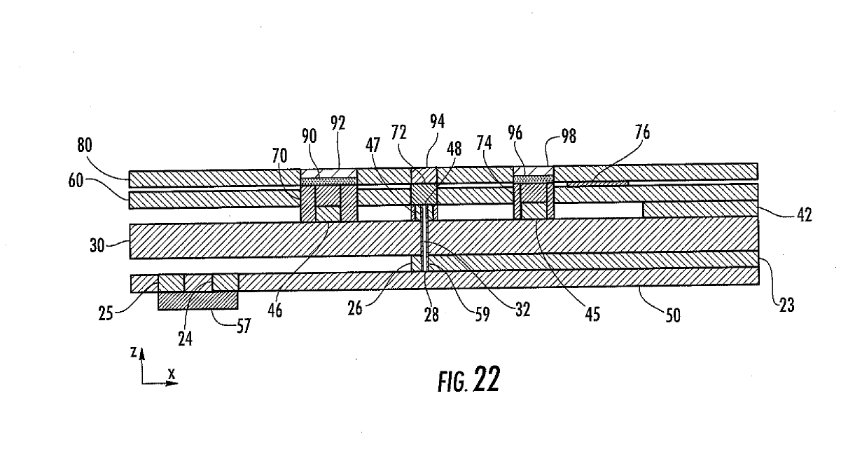

As shown in FIG. 22, membrane layers can be applied through openings 82, 84, 86 and 88. In some embodiments, membrane layers 90 and 92 can be applied to opening 82, a membrane layer 94 can be applied to opening 84, membrane layers 96 and 98 can be applied to opening 86, and membrane layer 100 can be applied to opening 88. FIG. 22 is illustrated such that membrane layers 90 and 92 and 96 and 98 produce working electrodes, membrane layer 94 produces a reference electrode, and membrane layer 100 forms a counter electrode, although other configurations and numbers of electrodes could be used. The working electrode(s) can be used to measure the concentration of a particular redox reactive species, which can then be used to determine the concentration of a particular analyte. The reference electrode establishes a fixed potential from which the potential of the counter electrode and the working electrode can be established. The counter electrode provides a working area for conducting the majority of electrons produced from the oxidation chemistry back to the solution.

The membrane layers 90 and 96 can be redox reactive membrane layers and include a redox reactive species for use in detecting an analyte in a fluid. For example, membrane layers 90 and 96 can include a redox reactive species such as glucose oxidase for detecting glucose. The membrane layers 90 and 96 can also include a redox mediator, carbon nanostructures, or other suitable materials. Suitable membrane layers are described, for example, in U.S. application Ser. No. 12/436,013, filed May 5, 2009 and this application is incorporated by reference in its entirety. In some embodiments, both membrane layers 90 and 96 can include a redox reactive species for a particular analyte (e.g. glucose oxidase for glucose). In these embodiments, both membrane layers 90 and 96 can produce measurements of analyte concentration and can be averaged to provide a more accurate measurement of the analyte concentration. In some embodiments, one of the membrane layers (e.g. 90) can be a redox reactive membrane layer and can include a redox reactive species for a particular analyte and the other membrane layer (e.g. 96) can be provided without a redox reactive species. In such a configuration, the membrane layer 96 can form an interference membrane and can be used to measure the concentration of interfering analytes in the fluid of interest that may produce electrons. For example, the redox reactive membrane layer 90 can measure glucose concentration and the interference membrane layer 96 can measure the current produced by an interfering species such as acetaminophen. The measurement made from the redox reactive membrane layer 90 can be adjusted based on the measurement made from the interference membrane layer 96 to provide a more accurate measurement of analyte concentration. The membrane layers 92 and 98 provided on top of the membrane layers 90 and 96 may or may not be present and can be a polymeric material such as ethylene vinyl acetate (EVA) copolymer. The membrane layers 92 and 98 can be used to selectively allow the passage of analytes including the analyte of interest to the membrane layers 90 and 96.

Membrane layer 94 for the reference electrode can be a formed of a conductive material. In some embodiments, the membrane layer 94 is an ion-sensitive electrode comprising a metal/metal halide layer such as silver/silver chloride. Membrane layer 100 for the counter electrode may or may not be present and can be a polymeric material such as ethylene vinyl acetate (EVA) copolymer. It is noted that membrane layer 100 is offset from the centerline 2-2 in the y-direction and thus is not illustrated in FIG. 22.

In some embodiments, the flexible circuit 10 forms an amperometric sensor, wherein a redox voltage is applied and a current is generated that is generally proportional to the amount of the redox reactive species in the liquid test sample. Although FIG. 22 is depicted including two working electrodes, a reference electrode and a counter electrode, the flex circuit 10 can include any configuration for a sensor and generally will include from 2-6 electrodes. In some embodiments, the flex circuit 10 includes at least one working electrode, a counter electrode and a reference electrode.

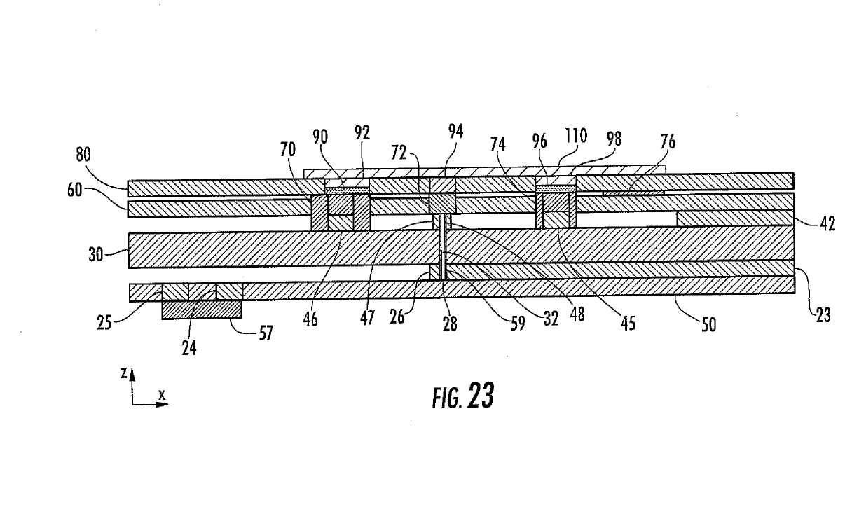

As illustrated in FIG. 23, a polymeric material 110 that allows for the passage of the analyte being measured can optionally be applied to the top surface of the flexible circuit 10 to cover membrane layers 92, 94, 98 and 100. The polymeric material 110 can allow molecules to pass at a certain rate so the sensor can accurately measure the analyte in a fluid of interest, for example, the glucose level in blood. The polymeric material 110 can also prevent the membrane layers or conductive material from leaching into the fluid of interest. In some embodiments, the polymeric material can be an ethylene vinyl acetate (EVA) copolymer. Although the polymeric material 110 in FIG. 23 completely covers the membrane layers 92, 94, 98 and 100, the polymeric material can be applied such that it only covers some of the electrodes or a portion of a particular electrode, particularly if a polymeric membrane layer is also provided as a membrane layer.

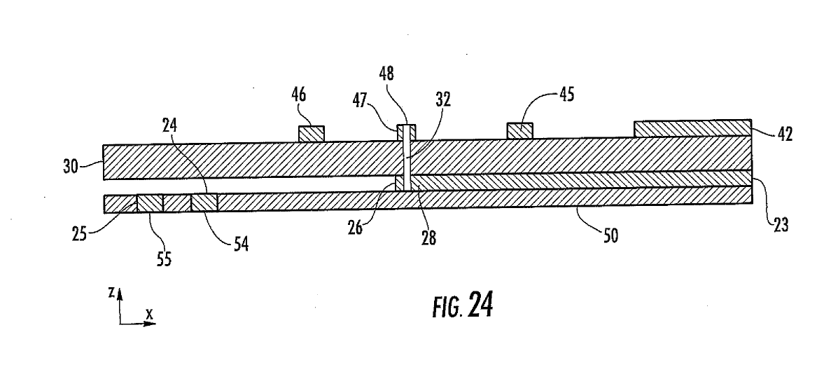

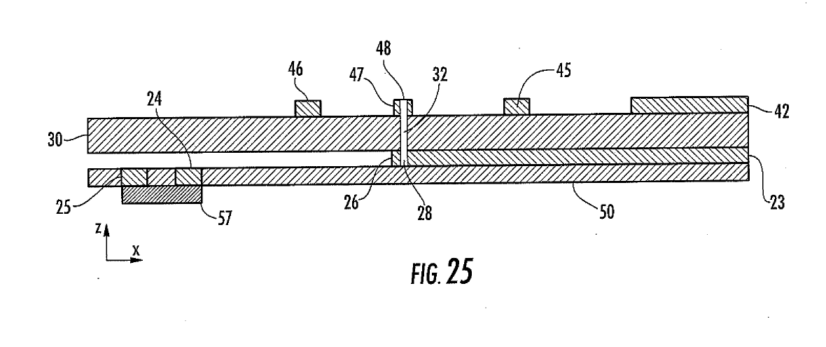

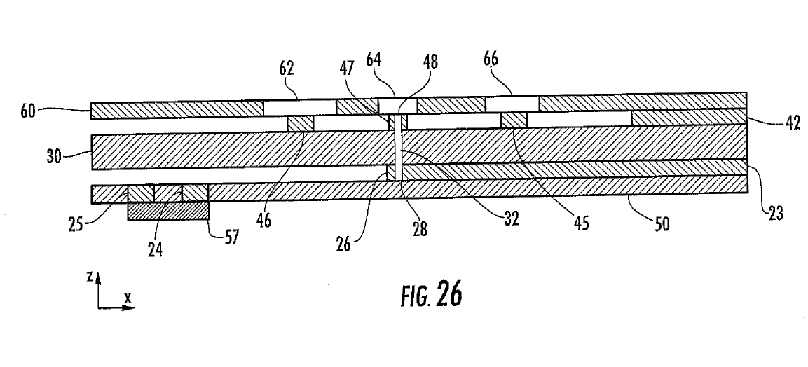

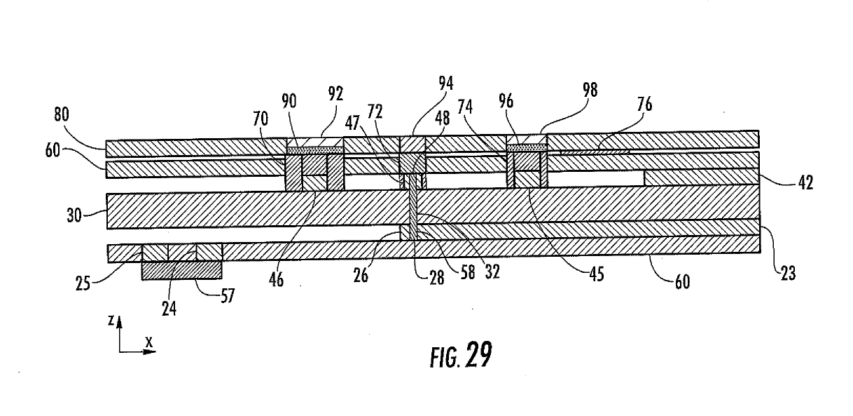

In another embodiment illustrated in FIGS. 24-30, a via 58 can be produced by a different process. In particular, holes such as holes 28, 32 and 48 can be formed in the bottom metal layer 20, dielectric substrate 30 and top metal layer 40 as described above with respect to FIG. 15. Instead of forming a via 59 as described above with respect to FIG. 16, the holes 28, 32 and 48 can be maintained through the application of the bottom mask layer 50 and the top mask layer 60 as shown in FIGS. 24-26. The via 58 can be formed when the conductive ink layer 72 is applied through the opening 64 corresponding to via 58. In particular, by selecting a conductive ink for conductive ink layer 72 that has a viscosity and cohesive and adhesive properties for the size of the holes 28, 32 and 58, that allows the ink to flow into and fill the holes, the via 58 can be formed. The conductive ink can be a conductive fluid or a conductive solution and, in some embodiments, can be a platinum/graphite ink or a silver/silver chloride ink. In some embodiments, the conductive ink is applied by screen printing. FIG. 27 illustrates the formation of the via 58 using conductive ink 72. The flex circuit 10 can be prepared in the same manner described above once the via 58 is formed as shown in FIGS. 28-30.

FIG. 31 provides a plan view of the top surface 112 of the flexible circuit 10. The flexible circuit 10 includes the dielectric substrate 30 and membrane layers 92, 94, 98 and 100 provided on the dielectric substrate 30 on the top surface 112 of the flexible circuit. In some embodiments, the dielectric substrate 30 can be between about 0.02 and 0.06 inches wide and between about 1.0 and 3.0 inches long. The width can also be from 0.01 to 0.02 inches wide as discussed herein because the use of vias such as via 58 or 59 can reduce the width needed for wiring of the flexible circuit by as much as one half or more if additional metal layers are used in the flex circuit 10. Alternatively, the flex circuit 10 can support twice as much wiring for a given width, or more if additional metal layers are used. In FIG. 22, the electrical wires 41, 42 and 43 can communicate with the membrane layers 100, 94 and 98, respectively, as described herein. Electrical wire 23 can communicate with membrane 94 (through conductive ink layer 72 and via 48 or 49).

As shown in FIG. 31, the membrane layer 100 corresponding to the reference electrode can communicate with an underlying contact 44 that is offset from the membrane layer 100 in the y-direction through the use of conductive ink layer 76. The distance of the offset can vary depending on the particular application and the arrangement and configuration of the electrodes. In some embodiments, the distance of the offset can be from 0.003 to 0.050 inches. The use of the offset prevents the electrolytes present in the fluid of interest from contacting the underlying metal contact (i.e. contact 44) and oxidizing it. Thus, the underlying metal contact can be formed of a cheaper material such as copper or a copper alloy. Further, spacing the contact 44 from the membrane layer 100 also enhances diffusion resistance. The offset can also prevent the underlying contact 44 from oxidizing at a positive potential, such as would be the case for a glucose electrode measuring peroxide vs. silver-silver chloride. This type of configuration and the benefits thereof are described in published U.S. Patent Appl. Nos. 2007/0200254 and 2007/0202672, which are hereby incorporated by reference in their entirety.

FIG. 32 illustrates the bottom surface 114 of the flex circuit 10. As shown in FIG. 32, a thermistor 57 can be provided on the bottom surface 114 of the flex circuit 10. The thermistor 57 can be in electrical contact with contacts 24 and 25 and thus with wires 21 and 22, respectively. Contact 26 and via 58 or 59 are in electrical communication with wire 23 and in the z-direction with conductive ink layer 72.

Through the use of the vias such as via 58 or 59, the flexible circuit 10 can have wiring on a separate metal layer, such as bottom metal layer 20 adjacent the bottom surface 114 of the flexible circuit, thus allowing for a reduction in the amount of wiring that occurs in a single metal layer, such as the top metal layer 40 adjacent the top surface 112. Thus, the flexible circuit 10 can be constructed with a narrower profile in the y-direction and thus can be more easily incorporated in a medical instrument such as within the lumen wall of a catheter. In some embodiments, the placement of the via 58 or 59 in direct communication with an electrode (e.g. the reference electrode at membrane layer 94) instead of having the via formed in wiring communicating with the electrode can be used to reduce the wiring that is needed in a particular metal layer. The wiring communicating with the electrode communicating with the via (e.g. the reference electrode at membrane layer 94) can be provided in a separate metal layer (e.g. bottom metal layer 20) instead of in the metal layer used for other electrodes (e.g. top metal layer 40). In other words, in some embodiments, no wiring for the electrode communicating with the via will be provided in the metal layer used for the other electrodes. As a result, the width of the flex circuit 10 for a given number of electrodes can be reduced. In some embodiments, the via 58 or 59 is directly below the electrode (e.g. the reference electrode at membrane layer 94) such that an axis drawn through the center of the via 58 or 59 intersects with the membrane layer (e.g. 94).

Although the flexible circuit 10 provided in the figures includes two metal layers 20 and 40, additional metal layers can be separated by a dielectric layer and can be connected electrically through the use of one or more additional vias through the dielectric layer like via 58 or 59 to provide additional contacts and wiring in the flex circuit and to allow for a further reduction of width in the flex circuit 10. For example, the flex circuit 10 could include a metal/dielectric/metal/dielectric/metal construction as an alternative to the metal/dielectric/metal construction provided in the figures. In addition, more than one via can provide electrical communication between the top metal layer 40 and the bottom metal layer 20. Vias can also be provided that allow electrical communication between electrodes present on the bottom surface 114 of the flex circuit and contacts and wiring provided in the top metal layer 40.

The flex circuit 10 described herein is a two-sided flex circuit, with metal layers 20 and 40 provided on opposing sides of a dielectric substrate 30. The flex circuit 10 can have electrodes provided on opposing sides (e.g., the working, reference and counter electrodes on the top surface 112 and the thermistor on the bottom surface 114). In some embodiments, the flex circuit 10 can include offset portions on the top and bottom surfaces of the dielectric substrate used in the flex circuit. This can be accomplished, for example, by taking the offset arrangement characterized by contact 44, conductive ink layer 76 and membrane layer 100 on the top surface 112 of the flex circuit 10 and creating corresponding structures on the bottom surface 114.

As described herein, the wires 23, 41, 42 and 43 can be connected to the measurement device. In some embodiments, the wires 23, 41, 42 and 43 transmit power to the electrodes for sustaining an oxidation or reduction reaction, and can also carry signal currents to a detection circuit (not shown) indicative of a parameter being measured. In some embodiments, the parameter being measured can be any redox reactive species that occurs in, or can be derived from, blood chemistry. For example, the redox active chemical species can be hydrogen peroxide, formed from reaction of glucose with glucose oxidase, thus having a concentration that is proportional to blood glucose concentration. Although not illustrated, the flexible circuit 10 can be designed to terminate to a tab that mates to a multi-pin connector, such as a 3-pin, 1 mm pitch ZIF Molex connector. Such a connection facilitates excitation of the working electrode and measurement of electrical current signals, for example, using a potentiostat or other controller.

The flex circuit 10 can be incorporated into a tubular medical instrument such as a catheter or an intraocular implant. Such a design can, for example, facilitate utilization of the flex circuit 10 for invasively monitoring blood glucose levels. For example, a catheter can include a tubular body defining one or more lumens. The flex circuit 10 can be positioned in the catheter wall such that the top surface 112 of the flex circuit can be exposed to the environment outside of the catheter for contact with the blood stream (or other fluid of interest) and the bottom surface 114 can be exposed to a lumen of the catheter. In one embodiment, the flex circuit is attached to a lumen wall via an adhesive. One method of doing this is described in published U.S. Patent Appl. No. 2009/0024015, which is hereby incorporation by reference in its entirety.

The “two-sided” flexible circuit provided herein that comprises conductive material on the top and bottom planar surfaces of a dielectric substrate can have its reference electrode substituted with or be part of a system comprising the reference electrode channel described above and as shown as in FIGS. 1-12. In addition, the reference electrode can be configured as a counter electrode and used together with the two-sided flexible circuit provided herein. Thus, the two-sided flex circuit can comprise a reference/counter electrode that is masked and imaged onto the substrate or a separate substrate.

Many modifications and other embodiments will come to mind to one skilled in the art having the benefit of the teachings presented in the foregoing description and the associated drawings. For example, while the disclosure has generally described exemplary embodiments as including a flexible circuit, the invention may also be used in conjunction with stiffer substrates. Therefore, it is to be understood that modifications and other embodiments are intended to be included within the scope of the appended claims. Although specific terms are employed herein, they are used in a generic and descriptive sense only and not for purposes of limitation.

That which is claimed:

1. A sensor including a flexible circuit, comprising:
a flexible dielectric substrate having opposing first and second planar surfaces defining longitudinal, transverse and normal directions;
one or more conductive contacts adjacent the first planar surface of said flexible dielectric substrate;
one or more conductive contacts adjacent the second planar surface of said flexible dielectric substrate;
a first dielectric mask adjacent the first planar surface and substantially covering the first planar surface, said first dielectric mask having one or more mask openings corresponding to one of more of the conductive contacts adjacent the first planar surface;
a second dielectric mask adjacent said second planar surface substantially covering said second planar surface;
at least one conductive material provided within the mask openings of said first dielectric mask and in electrical communication with the one or more conductive contacts adjacent the first planar surface;
a via extending through said dielectric substrate and providing electrical communication between a contact adjacent the first planar surface and a contact adjacent the second planar surface to provide electrical communication between the conductive material within one of the mask openings and the contact adjacent the second planar surface;
wires in electrical communication with the contacts adjacent the first planar surface and the contacts adjacent the second planar surface; and
one or more membrane layers applied in physical contact with at least a portion of the conductive material, said one or more membrane layers performing a chemical transduction that is communicated to the conductive material.

2. The sensor according to claim 1, wherein the second dielectric mask includes one or more mask openings corresponding to one or more conductive contacts adjacent the second planar surface and further comprising a conductive material applied to the second dielectric mask adjacent the mask openings in the second dielectric mask such that the conductive material is in electrical connection with the one or more conductive contacts adjacent the second planar surface.

3. The sensor according to claim 2, wherein the conductive material applied to the second dielectric mask adjacent the mask openings in the second dielectric mask is in electrical communication with two conductive contacts adjacent the second planar surface and forms a thermistor with the two conductive contacts.

4. The sensor according to claim 1, further comprising a third dielectric mask adjacent the first dielectric mask and substantially covering the first dielectric mask, said third dielectric mask having one or more mask openings corresponding to one of more of the conductive contacts adjacent the first planar surface, at least a portion of said one or more membrane layers provided within the mask openings in the third dielectric mask.

5. The sensor according to claim 1, wherein at least one contact and at least one membrane layer corresponding to the at least one contact are offset from one another and are in communication with each other through the at least one conductive material provided in the mask openings of the first dielectric mask.

6. The sensor according to claim 5, wherein the at least one contact and the at least one membrane layer corresponding to the at least one contact are offset from one another in a transverse direction.

7. The sensor according to claim 1, wherein the at least one conductive material applied to at least one of the mask openings of the first dielectric mask is different than the conductive material applied to another of the at least one of the mask openings of the first dielectric mask.

8. The sensor according to claim 1, wherein said via is hollow.

9. The sensor according to claim 1, wherein said via is solid.

10. The sensor according to claim 1, wherein said via includes a layer of nickel and a layer of gold.

11. The sensor according to claim 9, wherein said via is formed by the conductive material applied to the mask openings in the first dielectric mask.

12. The sensor according to claim 1, wherein said via is directly below the conductive material with which it is in electrical communication.

13. The sensor according to claim 1, wherein said conductive material is a metal/metal halide layer and one or more of the membrane layers form an ion sensitive electrode.

14. The sensor according to claim 1, wherein the one or more membrane layers form a working electrode, a counter electrode, and a reference electrode.

15. A sensor for measuring the concentration of a redox reactive species in a fluid of interest, comprising:
a flexible dielectric substrate having opposing top and bottom planar surfaces defining longitudinal, transverse and normal directions;
a working electrode comprising a membrane material including a redox reactive species and an underlying conductive material, said underlying conductive material in electrical communication with a conductive contact adjacent the top planar surface of said dielectric substrate;
a counter electrode comprising a conductive material in electrical communication with a conductive contact adjacent the top planar surface of said dielectric substrate;
a reference electrode comprising a conductive material in electrical communication with a conductive contact adjacent the top planar surface of said dielectric substrate;
a bottom contact comprising a conductive material adjacent the second planar surface of said dielectric substrate; and
a via extending in electrical communication with one of said working electrode, said counter electrode and said reference electrode and the bottom contact through said dielectric substrate along a normal direction to provide a conductive path between one of said working electrode, said counter electrode and said reference electrode and said bottom contact.

16. The sensor according to claim 15, further comprising:
a first trace in electrical communication with said working electrode;
a second trace in electrical communication with said counter electrode;
a third trace in electrical communication with said reference electrode;
wherein the trace in electrical communication with the one of said working electrode, said counter electrode and said reference electrode that is in electrical communication with said bottom contact is provided adjacent the second planar surface of said dielectric substrate and the other traces are provided adjacent the first planar surface of said dielectric substrate.

17. A method for producing a flexible circuit, comprising:
providing a substantially planar, flexible dielectric substrate having opposing first and second planar surfaces having longitudinal, transverse and normal directions;
forming at least one first conductor layer adjacent the first planar surface of the dielectric substrate, said first conductor layer comprising one or more contacts and one or more wires;
forming at least one second conductor layer adjacent the second planar surface of the dielectric substrate, said second conductor layer comprising one or more contacts and one or more wires;
forming a hole in the normal direction through the first conductor, the dielectric substrate and the second conductor;
depositing conductive material within the hole of the dielectric substrate to provide a conductive path extending through the dielectric substrate in a normal direction, wherein the conductive path is in electrical communication with the first conductor and the second conductor;
forming a first dielectric mask adjacent the first planar surface and substantially covering the first planar surface, the first dielectric mask having one or more mask openings corresponding to said at least one first conductor;
forming a second dielectric mask adjacent the second planar surface substantially covering the second planar surface;
depositing at least one conductive material within the mask openings of the first dielectric mask in electrical communication with the at least one conductor adjacent the first planar surface; and
depositing one or more membrane layers in physical contact with at least a portion of the conductive material.

18. The method according to claim 17, wherein forming a second dielectric mask includes forming a second dielectric mask comprising one or more mask openings corresponding to one or more contacts adjacent the second planar surface, said method further comprising applying a conductive material to the second dielectric mask adjacent the mask openings in the second dielectric mask such that the conductive material is in electrical connection with the one or more contacts adjacent the second planar surface.

19. The method according to claim 18, wherein the conductive material applied to the second dielectric mask adjacent the mask openings in the second dielectric mask is in electrical communication with two conductive contacts adjacent the second planar surface and forms a thermistor with the two conductive contacts.

20. The method according to claim 17, further comprising forming a third dielectric mask adjacent the first dielectric mask and substantially covering the first dielectric mask, the third dielectric mask having one or more mask openings corresponding to one of more of the contacts adjacent the first planar surface, at least a portion of said one or more membrane layers provided within the mask openings in the third dielectric mask.

21. The method according to claim 17, wherein at least one contact and at least one membrane layer corresponding to the at least one contact are offset from one another and are in communication with each other through the at least one conductive material provided in the mask openings of the first dielectric mask.

22. The method according to claim 21, wherein the at least one contact and the at least one membrane layer corresponding to the at least one contact are offset from one another in a transverse direction.

23. The method according to claim 17, wherein the at least one conductive material applied to at least one of the mask openings of the first dielectric mask is different than the conductive material applied to another of the at least one of the mask openings of the first dielectric mask.

24. The method according to claim 17, wherein depositing conductive material within the hole of the dielectric substrate comprises depositing conductive material to foam a hollow via.

25. The method according to claim 17, wherein depositing conductive material within the hole of the dielectric substrate comprises depositing conductive material to form a solid via.

26. The method according to claim 17, wherein depositing conductive material within the hole of the dielectric substrate comprises electroplating metal inside the hole.

27. The method according to claim 17, wherein depositing conductive material within the hole of the dielectric substrate comprises plating nickel via an electroless plating process and plating gold via an immersion plating process within the hole.

28. The method according to claim 27, wherein depositing at least one conductive material within the mask openings of the first dielectric mask comprises depositing conductive material within the hole of the dielectric substrate to form a conductive path.

29. The method according to claim 17, wherein depositing at least one conductive material within the mask openings of the first dielectric mask comprises depositing at least one conductive material directly above the hole formed through the first conductor, the dielectric substrate and the second conductor.

30. The method according to claim 17, wherein depositing one or more membrane layers comprises depositing membrane layers forming a working electrode, a reference electrode and a counter electrode, wherein at least one of the working electrode, the reference electrode and the counter electrode is in electrical communication with the conductive path through the hole.

31. The method according to claim 30, wherein depositing one or more membrane layers comprises depositing a membrane layer comprising a redox reactive species and forming at least a portion of the working electrode.

32. The method according to claim 31, wherein the redox reactive species is an enzyme for use in detecting glucose concentration.

33. The method according to claim 17, wherein the conductive material is deposited within the hole prior to forming the first dielectric mask and forming the second dielectric mask.

34. A medical instrument, comprising:
a tubular body defining at least one lumen; and
a flexible circuit positioned in said tubular body, the flexible circuit including:
a flexible dielectric substrate having opposing first and second planar surfaces defining longitudinal, transverse and normal directions;
one or more conductive contacts adjacent the first planar surface of said flexible dielectric substrate;
one or more conductive contacts adjacent the second planar surface of said flexible dielectric substrate;
a first dielectric mask adjacent the first planar surface and substantially covering the first planar surface, said first dielectric mask having one or more mask openings corresponding to one of more of the conductive contacts adjacent the first planar surface;
a second dielectric mask adjacent said second planar surface substantially covering said second planar surface;
at least one conductive material provided within the mask openings of said first dielectric mask and in electrical communication with the one or more conductive contacts adjacent the first planar surface;
a via extending through said dielectric substrate and providing electrical communication between a contact adjacent the first planar surface and a contact adjacent the second planar surface to provide electrical communication between the conductive material within one of the mask openings and the contact adjacent the second planar surface;
wires in electrical communication with the contacts adjacent the first planar surface and the contacts adjacent the second planar surface; and
one or more membrane layers applied in physical contact with at least a portion of the conductive material, said one or more membrane layers forming a working electrode, a reference electrode and a counter electrode, wherein at least one of the working electrode, the reference electrode and the counter electrode is in electrical communication with said via.
